(19) United States
(12) Patent Application Publication
(10) Pub. No.: US 2009/0171290 A1
(43) Pub. Date: Jul. 2, 2009

(54) INFUSION DEVICE FOR INFUSING MULTIPLE MEDICAMENTS AT DIFFERENT SPEEDS AND METHOD

(75) Inventors: Nan Li, Shanghai (CN); Shuqun Cheng, Shanghai (CN); Hao Li, Shanghai (CN)

Correspondence Address: KIRTON AND MCCONKIE, 60 EAST SOUTH TEMPLE,, SUITE 1800, SALT LAKE CITY, UT, 84111, US

(73) Assignee: Nan Li, Shanghai (CN)

(21) Appl. No.: 12/125,321

(22) Filed: May 22, 2008

(30) Foreign Application Priority Data
Dec. 28, 2007 (CN) 200710173488.8

(51) Int. Cl. A61M 5/142; A61M 5/32

(52) U.S. Cl. 604/151; 604/174

(57) ABSTRACT

An infusion device comprises a pump having first and second chambers that are sealed by a penetrable membrane; an injection catheter comprises a first passageway which is connected to the first chamber; and an expandable elastic pouch which is mounted to the injection catheter and is connected to the second chamber through inlet holes defined in a wall of the second passageway. The expandable elastic pouch further connects an outlet port of the injection catheter through some outlet holes which are also defined in the wall. Medicaments are filled into the chambers and can either be directly injected through to the first passageway, or transmitted to the elastic pouch. The elastic pouch is expanded, and accordingly, a pressure is induced on the medicament to effectively continuous deliver the medicament for a given period of time.

### TECHNICAL FIELD OF THE INVENTION

The present invention relates to an infusion device for medical use, and particularly, to an infusion device for infusing multiple medicaments at different speeds and method thereof.

### BACKGROUND OF THE INVENTION

Conventionally, the medicament infusion pumps are embedded subcutaneously and are widely used to provide medicaments continuously for a period of time, in chemotherapy or relieving pain of the patients. Medicaments which are provided in the afore-said manner would make good curative effects and have fewer side-effects. Nevertheless, a continuous delivery of lower concentration medicaments is often needed to conjunct with higher dose medicaments injection in the clinical and medical practices and application, and a prior chemotherapy pump cannot fulfill this purpose. Consequently, the present invention is aimed to provide an infusion device with two chambers desirable as a response to the actual requirements in clinical and medical services. The infusion device can be completely embedded in a human body, or the pump of the infusion device can be left outside the human body with the injection catheter of the infusion device embedded in the body cavity or blood vessels. The present invention infusion device is more adaptive for the actual requirements and has more competition in the market.

### SUMMARY OF THE INVENTION

For solving the problem of known prior art chemotherapy pump being incapable of effecting injection of higher dose medicaments while continuously deliver lower concentration medicaments, the present invention provides an infusion device for selectively infusing various medicaments at different speeds. The infusion device is not only capable of attaining the curative amount rapidly through one time injection of higher dose medicaments, but also capable of keeping the consistency of continuously infused medicament at a constant value. Thus, the present invention infusion device enhances the curative effects as well as simplifies the operation. Accordingly, the inconvenience of long infusion time can be avoided, and thus, the patients' quality of life can be effectively enhanced.

In one aspect, the present invention provides an infusion device for infusing multiple medicaments at different speeds; the infusion device at least comprises: a pump having a first chamber, a second chamber, an outlet tube, and a top surface having a penetrable membrane covering the first chamber and second chamber; an injection catheter including an inlet port, an outlet port, a first passageway which is connected to the first chamber, and a second passageway; a high-resilient elastic pouch mounted to the injection catheter connected to the second chamber through a plurality of inlet holes defined in a wall of the second passageway and connected to the outlet port of the injection catheter through a plurality of outlet holes also defined in the wall of the second passageway.

In another aspect, the present invention provides an infusion device for infusing multiple medicaments at different speeds; the infusion device at least comprises: a pump having a plurality of chambers, an outlet tube, and a top surface having a penetrable membrane covering the chambers; an injection catheter having an inlet port, an outlet port, and a plurality of passageways, wherein at least one passageway is connected to one of the chambers; and a plurality of high-resilient elastic pouches mounted to the injection catheter and each connecting to a respective one of the chambers through a plurality of inlet holes defined in a wall of the second passageway and connecting to the outlet port of the injection catheter through a plurality of outlet holes also defined in the wall.

In yet another aspect, the present invention provides a method of infusing multiple medicaments at different speeds, also capable of injecting higher dose medicaments and continuously delivering lower concentration medicaments by using an infusion device. The infusion device comprises a pump having a first chamber, a second chamber, an outlet tube, and a top surface having a penetrable membrane covering the first chamber and second chamber; an injection catheter having an inlet port, an outlet port, a first passageway which is connected to the first chamber, and a second passageway; and a high-resilient elastic pouch which is mounted to the injection catheter and connected to the second chamber through a plurality of inlet holes defined in a wall of the second passageway, and also connected to the outlet port through a plurality of outlet holes defined in the wall.

While the method of infusing multiple medicaments at different speeds according to the present invention is put into practice, the injection catheter is embedded subcutaneously, and the pump is either embedded subcutaneously or positioned outside a human body. If the pump is embedded subcutaneously, owing to a coating of aliphatic polycarbonate polyurethanes having good biocompatibility and elasticity on an outer surface of the infusion device, the occurrences of the rejection reactions can be avoided. On the opposite ends of the elastic pouch whereby connected to the injection catheter and the pump, the medicaments can be introduced into the first chamber through the penetrable membrane and directly infused into the objective body cavity or blood vessels if an injection of a higher dose medicament is needed while continuously deliver lower concentration medicament. The medicaments can be filled into the second chamber and then transmitted into the elastic pouch which is expanded to induce pressure on the medicament so as to infuse the medicament into the objective body cavity or blood vessels through the outlet port of the injection catheter.

Additionally, the end of the injection catheter is provided with an anti-backflow device for avoiding the occurrence of a reverse flow of the medicament and/or body fluids.

More specifically describing the invention, the aforementioned modes can be realized by the following steps:

First, a pump that is made from hard plastics constitutes an infusion device having two or more chambers according to the present invention, and then, a coating of aliphatic polycarbonate polyurethanes is set on the outer surface of the pump. The pump is shaped as two-connected circles and having a flat bottom and a smooth outer perimeter, also having a long axis of, for example, 4.0 cm, a short axis of, for example, 2.4 cm, and a height of, for example, 1.2 cm. Further, a penetrable membrane is set on the pump to seal the chambers. The penetrable membrane has a long axis of, for example, 3.0 cm and a short axis of, for example, 1.8 cm. The chambers include a front chamber and a rear chamber, where the front chamber is connected to an upper passageway of an injection catheter of the infusion device, and the rear chamber is connected to a lower passageway of the injection catheter through a high-resilient elastic pouch.

The high-resilient elastic pouch of the infusion device can be made of, for example, medical grade silicone rubber. The elastic pouch is covered with a coating of aliphatic polycarbonate polyurethanes and is connected to the front chamber of the pump through a plurality of holes defined in a wall of the upper passageway of the injection catheter. The size of the high-resilient elastic pouch is selectively set according to the therapeutic requirement of treatments. And the volume of the elastic pouch can be set to be 100 ml to 200 ml, for example.

Additionally, the part of the injection catheter between inlet holes and outlet holes of the elastic pouch is blocked so that medicament flowing through the upper passageway is only allowed to transmit through the inlet holes, the elastic pouch, and the outlet holes, in sequence. According to the needed infusion speed, the diameter of the outlet holes can be measured by the following equation:

π(diameter of hole/2)²=(volume of the elastic pouch)/(infusion speed*delivery time)

As far as the injection catheter is concerned, the length of the injection catheter can be 35 cm, for example. A front end of the injection catheter is connected to a tube of the pump by a connector, and a rear end is mounted with an anti-backflow device which can be an elastic crack tip or a check valve, for example.

If the pump of the infusion device is positioned outside the human body, the injection catheter can be fixed on a puncture site by a clamp. If the pump and the elastic pouch are positioned beneath the skin, the shape of the infusion device can be different designs in accordance with the patients' sizes for portable convenience.

Advantageously, the medicament delivery infusion device is capable of not only injecting higher dose medicaments, but also delivering lower concentration medicaments continuously. The coating of the aliphatic polycarbonate polyurethanes on the outer surface of the infusion device is biocompatible and elastic, by which the occurrences of the rejection reactions can be avoided. The anti-backflow device can effectively prevent the injection catheter from being blocked due to a reverse flow, so as to ensure that the medicaments are delivered smoothly in the passageways. As an extra benefit and convenience, the manufacturing of the present invention is easy and low costs. Therefore, the present invention is more appropriate for the medical treatments.

In conclusion, the present invention is innovative in the material, structure, and manufacture, especially in the product's function. The present invention is practical and has a good market outlook.

### BRIEF DESCRIPTION OF THE DRAWINGS

FIG. 1 is a schematic sectional view showing an infusion device according to the present invention, which is made in the form of a double-chambered structure;

FIG. 2 is a top view of the infusion device of the present invention;

FIG. 3 is a schematic view showing a high-resilient elastic pouch of the infusion device in accordance with the present invention;

FIG. 4 shows the relationship between volume and flow rate of the infusion device according to the present invention;

FIG. 5A is a top view of a clamp that fixes a injection catheter of the infusion device to a human body according to the present invention; and

FIG. 5B is a cross-sectional view of the clamp of the infusion device according to the present invention.

### DETAILED DESCRIPTION OF THE PREFERRED EMBODIMENT

The present invention will be further described in detail in conjunction with the accompanying drawings and a preferred embodiment.

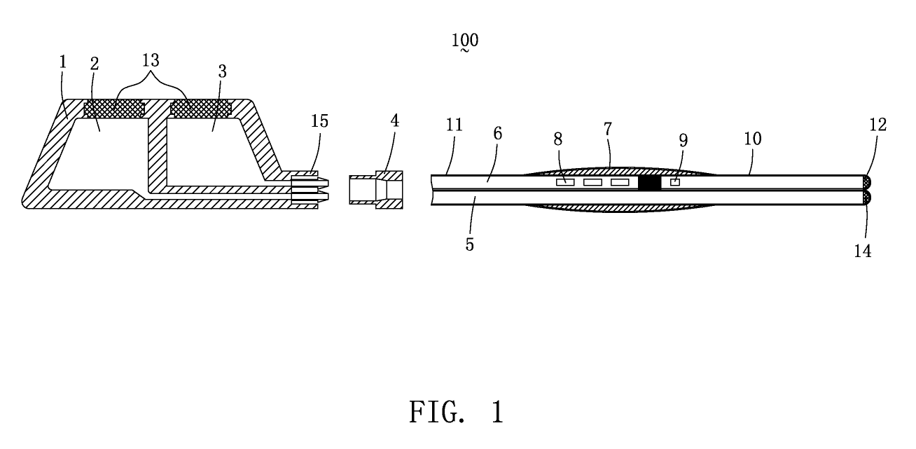

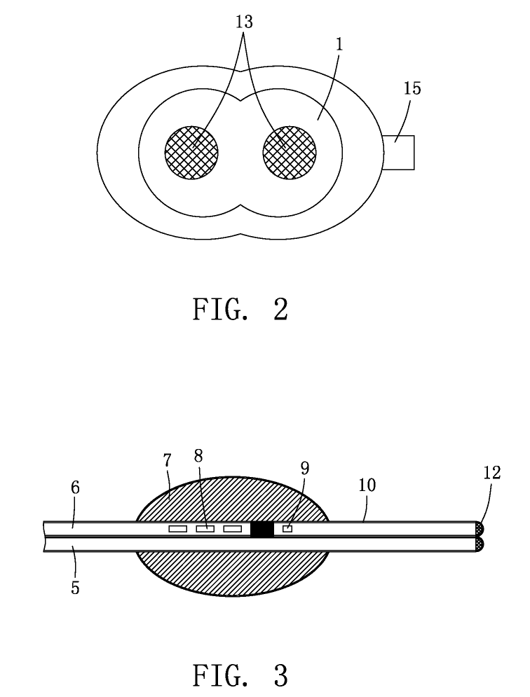

With reference to FIG. 1, which shows an infusion device constructed according to the preferred embodiment of the present invention, the infusion device is illustrated in the form of a double-cavity and double-passage delivery infusion device; however, it is noted that the illustration is not limitative to the scope of the present invention. The infusion device of the present invention comprises a pump 1 including a first chamber 2, a second chamber 3, an outlet tube 15, and a top surface carrying a penetrable membrane 13 covering each of the first chamber 2 and the second chamber 3; an injection catheter 10 having an inlet port 11, an outlet port 14, a first passageway 5 connected to the first chamber 2, and a second passageway 6; and a high-resilient elastic pouch 7 mounted to an outer surface of the injection catheter 10. The high-resilient elastic pouch 7 is connected to the second chamber 3 through a plurality of inlet holes 8 defined in a wall of the second passageway 6. The high-resilient elastic pouch 7 is further connected to the outlet port 14 through a plurality of outlet holes 9 defined in the wall of the second passageway 6.

The inlet port 11 of the injection catheter 10 is connected to the pump 1 by a connector 4. The number of the inlet holes 8 of the second passageway 6 is greater than that of the outlet holes 9.

An anti-backflow device 12 is arranged on the outlet port 14 of the injection catheter 10 to provide the passageways 5 and 6 with a one-way opening. The anti-backflow device 12 can be a simple elastic crack tip or a check valve, for example.

The infusion device 100 is coated with a layer of aliphatic polycarbonate polyurethanes.

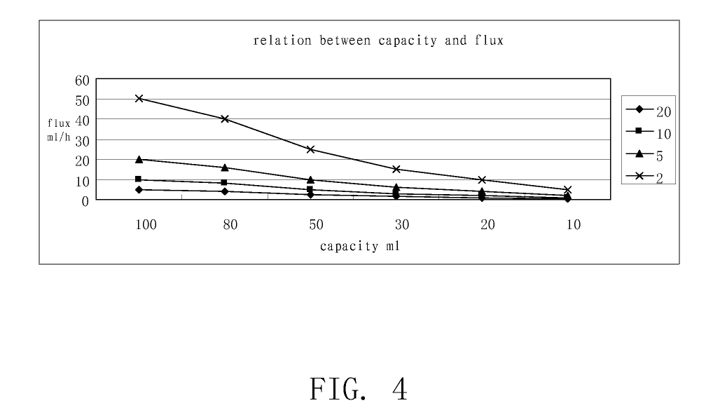

In practice, the infusion device according to the present invention is used in abdominal operations. The pump 1 is positioned under the skin of a patient and the injection catheter 10 and the high-resilient elastic pouch 7 are placed inside the abdomen. The inlet port 11 of the injection catheter 10 punctures from the belly and connects with the pump 1, and the outlet port 14 of the injection catheter 10 is set in a vessel or the abdomen. If an injection of higher dose medicament is needed, the medicament is introduced into the first chamber 2 through the penetrable membrane 13, and then the medicament is infused into the objective body cavity or blood vessels through the first passageway 5 to affect therapeutic treatment. And, if a continuous delivery of lower concentration medicament is needed, the medicament is introduced into the second chamber 3 and transmitted to the high-resilient elastic pouch 7 through the inlet holes 8 by which the elastic pouch 7 is expanded to accommodate the medicament. The medicament received in the elastic pouch 7 is then subjected to the pressure induced by the resiliency of the elastic pouch 7 for continuously and slowly infusing into the objective body cavity or blood vessels through the outlet holes 9 and the one-way opening formed in the end of the second passageway 6. The relationship between volume and flow rate according to the present invention is shown in FIG. 4.

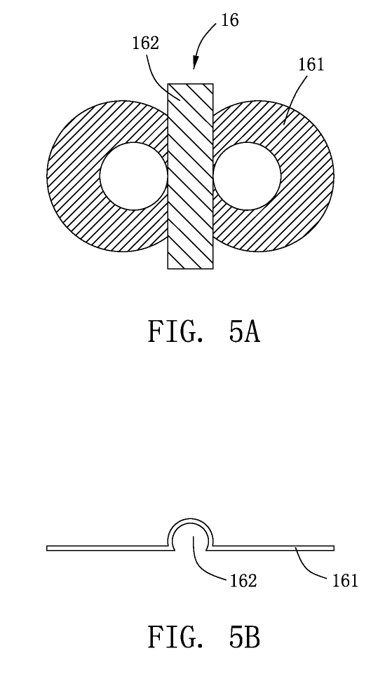

Alternatively, the injection catheter 10 can be made to puncture through the skin and the end of the injection catheter 10 is set in the body cavity or the vessel. The part of the injection catheter 10 outside of the body is fixed on the body surface by a clamp 16. Particularly shown in FIG. 5A and FIG. 5B, the clamp 16 has a “Ω” shape with a central injection catheter receiving slot 162 and two lugs 161 on opposite sides of the receiving slot 162. The injection catheter slot 162 accommodates a part of an injection catheter 10 and the lugs 161 are fixed on the human body to thereby fix the injection catheter 10 on the punctured site. In addition, the shapes of the pump 1 and the elastic pouch 7 can be of different shapes according to the size that is appropriate for the patients to be taking portably and for other curative services.

The aforementioned is just an example of the present invention which meets the need of the continuous lower concentration medicament delivery as well as the need of higher dose injections. If there are multiple medicaments, the present invention can be designed with several pumps and several injection catheters suitable for several elastic pouch assemblies. It is not intended to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

What is claimed is:

1. An infusion device comprising:
a pump having a first chamber and a second chamber, each of which has a penetrable membrane covering the first chamber and second chamber, and penetrable for filling first and second medicaments to the first and second chambers respectively;
an injection catheter having a first passageway connecting to the first chamber and a second passageway; and
an elastic pouch mounting to the injection catheter and connecting to the second chamber through a number of inlet holes defined in a wall of the second passageway and further connecting to an outlet port of the second passageway through a number of outlet holes defined in the wall of the second passageway.

2. The infusion device as claimed in claim 1, wherein the injection catheter has an inlet port connecting to an outlet tube of the pump by a connector.

3. The infusion device as claimed in claim 1, wherein the outlet port of the injection catheter is provided with an anti-backflow device.

4. The infusion device as claimed in claim 1, further comprising a coating of aliphatic polycarbonate polyurethanes on a surface of the infusion device.

5. The infusion device as claimed in claim 1, wherein the number of the inlet holes is greater than the number of the outlet holes of the second passageway.

6. The infusion device as claimed in claim 1, wherein the injection catheter further comprising a clamp adapted to fix the injection catheter to a human body.

7. An infusion device comprising:
a pump having a plurality of chambers and a penetrable membrane covering the plurality of chambers for filling medicaments to the chambers respectively;
an injection catheter having a plurality of passageways, at least one passageway is connecting to one of the chambers; and
a plurality of elastic pouches mounting to the injection catheter and each is connecting to a respective one of the chambers through a number of inlet holes defined in a wall of a respective one of the passageways and further connecting to an outlet port of said respective passageway through a number of outlet holes defined in the wall of the passageway.

8. The infusion device as claimed in claim 7, wherein the injection catheter has an inlet port connecting to an outlet tube of the pump by a connector.

9. The infusion device as claimed in claim 7, wherein the outlet port of the injection catheter is provided with an anti-backflow device.

10. The infusion device as claimed in claim 7 further comprising a coating of aliphatic polycarbonate polyurethanes on a surface of the infusion device.

11. The infusion device as claimed in claim 7, wherein the number of inlet holes defined in the wall of said respective passageway is greater than the number of outlet holes.

12. The infusion device as claimed in claim 7, wherein the injection catheter further comprises a clamp adapted to fix the injection catheter to a human body.

13. A method of infusing at least two medicaments at different speeds, also capable of injecting higher dose medicaments and continuously delivering lower concentration medicaments, the method comprising the following steps:
providing an infusion device comprising a pump having a first chamber and a second chamber sealed by a penetrable membrane, and an injection catheter having a first passageway connecting to the first chamber and a second passageway, while an elastic pouch is mounting to the injection catheter connecting to the second chamber through a number of inlet holes defined in a wall of the second passageway and further connecting to an outlet port of the injection catheter through a number of outlet holes also defined in the wall;
selectively filling a high dose medicament into the first chamber through the penetrable membrane for effectively directing injection of the high dose medicament through the first passageway; and
selectively filling the lower concentration medicament into the second chamber through the penetrable membrane and transmitting the lower concentration medicament to the elastic pouch through a number of inlet holes to thereby expanding the elastic pouch and thus inducing a pressure on the lower concentration medicament transmitting into the elastic pouch to continuously deliver the lower concentration medicament through the outlet holes and the outlet port of the injection catheter by the pressure induced on the elastic pouch.

14. The method as claimed in claim 13, wherein the injection catheter has an inlet port connected to an outlet tube of the pump by a connector.

15. The method as claimed in claim 13, wherein the outlet port of the injection catheter is provided with an anti-backflow device.

16. The method as claimed in claim 13, wherein the number of the inlet holes is greater than the number of the outlet holes of the second passageway.

17. The method as claimed in claim 13, wherein the injection catheter comprises a clamp adapted to fix the injection catheter to a human body.
